(19) United States
(12) Patent Application Publication
(10) Pub. No.: US 2021/0392870 A1
(43) Pub. Date: Dec. 23, 2021

(54) Apparatus for Heat Treatment of Motor Vehicles or Motor Vehicle Parts

(71) Applicant: UNIKAI LAGEREI- UND SPEDITIONSGESELLSCHAFT MBH, Hamburg (DE)

(72) Inventor: Patrick ALBERS, Hamburg (DE)

(73) Assignee: UNIKAI LAGEREI- UND SPEDITIONSGESELLSCHAFT MBH, Hamburg (DE)

(21) Appl. No.: 17/289,995

(22) PCT Filed: Dec. 4, 2018
(86) PCT No.: PCT/EP2018/083447

§ 371 Date: Apr. 29, 2021

(30) Foreign Application Priority Data
Nov. 1, 2018 (EP) 18203990.9

(51) Int. Cl. A01M 19/00; B65D 88/74

(52) U.S. Cl. CPC A01M 19/00; B65D 2588/743; B65D 88/745

(57) ABSTRACT

The invention relates to an apparatus for heat treatment of motor vehicles or motor vehicle parts, in particular for the purpose of killing pests, comprising art insulated sea freight container for temperature-controlled transport, the apparatus comprising at least one fan and at least one heat exchanger in addition to a cooling assembly which is possibly arranged in the container and is possibly capable of functioning.

The invention relates to an apparatus for heat treatment of motor vehicles or motor vehicle parts, in particular for the purpose of killing pests.

Heat treatment or thermal treatment of motor vehicles is understood to entail heating a motor vehicle in such a way that the motor vehicle exceeds a certain minimum temperature for a certain time at its coldest point. Such heat treatment can be used, in particular, to comply with legal or regulatory requirements that require pest control treatment of goods to be exported or imported. In particular, to control the brown marmorated stink bug (BMSB), Australia and New Zealand currently require that motor vehicles imported from certain countries during a specified period of the year be treated within 120 hours prior to departure of, for example, the vessel. Currently, it is recognized as sufficient to heat each motor vehicle in such a way that it has a temperature of at least 50 degrees Celsius (Australia) at its coldest point for a period of at least 20 minutes or a temperature of at least 56 degrees Celsius (New Zealand) for a period of at least 30 minutes. The treatment must be documented individually for each motor vehicle with all parameters. Owing to the size of the ships, up to 2,000 vehicles must be subjected to such treatment for one shipload.

One known practice is to carry out a heat treatment of this kind by equipping or building a warehouse for this purpose. However, this solution involves large investments and long approval periods under building law.

It is therefore the object of the invention to provide an apparatus for heat treatment of motor vehicles or motor vehicle parts, in particular for the purpose of killing pests, which has advantages over known solutions.

This object is achieved by an apparatus as claimed in claim 1. Particularly advantageous embodiments of the invention are the subject matter of the dependent claims.

An insulated sea freight container for temperature-controlled transport, also referred to as a refrigerated container, is a large-capacity container which has special thermal insulation compared to standard sea freight containers and which is typically used for temperature-sensitive cargo. In particular, there are refrigerated containers with an integrated refrigeration system (cooling unit), wherein the cooling unit is operated with electric power. Such refrigerated containers are also called integral containers. Refrigerated containers have a substantially rectangular basic shape with a long axis typically 40 feet (approx. 12 m) in length. The loading opening of the container is typically at one of the two ends, while the cooling unit is arranged at the other end, typically being inserted through a receiving opening. In this case, the cooling unit can be detachably secured by means of a circumferential flange with bolt connections around the outer edge of the receiving opening. Air cooled in the cooling unit can flow into the container interior. The interior floor of a refrigerated container can be designed with longitudinal ribs in such a way that longitudinal channels for cooling air are left between the ribs. Typically, air circulation within the container is enabled in such a way.

For the invention to work, it is not necessary that the container should have a refrigeration unit or that the refrigeration unit be functional.

The use of a refrigerated container has the advantage that used refrigerated containers can be obtained at reasonable cost. This applies especially when the refrigeration unit is faulty.

Owing to the already existing thermal insulation, energy efficiency per treated vehicle is high compared to a warehouse.

Compared to a warehouse equipped for heat treatment, the installation of the measuring sensors is significantly less costly with the solution according to the invention. Moreover, the installation of the ventilation systems for heating the interior of the motor vehicle is significantly less costly. In particular, the installation of an underfloor ventilation system is significantly less costly with the solution according to the invention.

In comparison with a warehouse equipped for heat treatment, which is typically significantly larger than a refrigerated container, a system consisting of a plurality of refrigerated containers has the further advantage that, because only a small number of motor vehicles, typically two, is heat treated in each refrigerated container, individual heat treatment of a small number of motor vehicles is possible without problems as regards minimum temperature and minimum duration.

The maximum capacity of a system consisting of a plurality of refrigerated containers is freely scalable. During the time when they are not in use, the containers can be stored or stacked efficiently, even outside the port, thereby avoiding useless occupation of expensive port areas. Since the conversion of a refrigerated container to an apparatus according to the invention represents only a minor intervention, it is also possible to reconvert the containers and use them for their original purpose again.

The proposed mobile hot water sources can be hired and set up without authorization. This eliminates capital costs for the time when they are not in use.

The at least one fan and the at least one heat exchanger are preferably arranged in the container. Energy losses through arrangement outside the container are thereby avoided.

The at least one fan and the at least one heat exchanger are preferably integrated in one unit. Such units are sold ready for use.

The container preferably comprises a cooling unit which can be operated by means of electric power and which comprises a refrigeration unit that is possibly functional, and at least one fan. The cooling unit fan, which is already present in any case, can then likewise be used to move and distribute the air. In this arrangement, the cooling unit is preferably arranged on one of the two ends of the container. The cooling unit preferably forms a vertical shaft on the inside of the end, wherein the rear wall of the cooling unit functionally complements the wall of the container or is identical therewith, wherein the transverse wall situated opposite the rear wall of the cooling unit has at least one through opening, through which air can flow out of the shaft into the container and/or out of the container into the shaft.

One of the substances which flow through the heat exchanger is preferably fed in through a line from outside the container and is discharged through a line to the outside of the container.

The motor vehicles are preferably passenger cars.

The at least one heat exchanger is preferably a water-air heat exchanger. The heat exchanger is preferably supplied by a mobile hot water source. The heat output of the heat exchangers is preferably at least 100 kW in total. The container preferably has at least one heat exchanger and one fan at each of the two ends. The apparatus preferably comprises two heat exchangers, the heat output of which is in each case at least 50 kW.

An inspection flap in the transverse wall of the cooling unit is preferably replaced by a plate which allows through a respective feed and return hose for each heat exchanger.

At least one pipe parallel to the longitudinal side of the container, having at least two outlets, can preferably distribute air in the container interior. It is particularly advantageous if a radial fan is mounted on the pipe. In this case, the outlets are preferably adjustable. The outlets are preferably arranged at uniform intervals. The at least one pipe is preferably an interlocked pipe. A respective pipe is preferably arranged in the region of the roof in the region of each longitudinal side wall. By means of these measures, it is possible to guide the air heated by a heat exchanger selectively also into regions of the motor vehicle to be heated, the heating of which would otherwise take longer.

Ribs that are arranged on the floor of the container and between which air can flow are partially covered, with the result that air flowing between the ribs can flow upward only through particular openings. In this process, air is forced into the spaces between the ribs by at least one fan arranged in the cooling unit. The openings are preferably adjustable. The openings are preferably arranged at regular intervals. These measures too allow selective guidance of heated air into regions of the motor vehicle to be heated, the heating of which would otherwise take significantly longer. The use of ribs which are present in any case in the floor of the refrigerated container and the use of a fan of the cooling unit makes installation of the underfloor ventilation system significantly easier than when equipping a warehouse for heat treatment.

An illumination device is preferably arranged in the container interior. There is preferably an emergency off switch.

The apparatus preferably has hinged access ramps at the loading opening. The loading ramps (access ramps) can preferably be connected detachably to the container. The loading ramps can preferably be secured detachably by means of at least one clamping device to cooling ribs on the floor of the container.

As a preferred possibility, relatively large items of freight or motor vehicles that are to be treated can be heated by combining a plurality of containers lengthwise, transversely and/or vertically.

At least one wired measuring sensor is preferably arranged in the container interior. Values measured by the measuring sensor or calculated on the basis of the measured values can preferably be communicated to a device suitable for documenting the values.

Preferably, when all the measured values have reached a predetermined limit value, the time during which all the values remain continuously in the predetermined range is measured, and, after the expiry of a predetermined minimum time, an optically or acoustically perceptible signal is produced.

The feed temperature for the at least one heat exchanger is preferably a maximum of 95 degrees Celsius.

A system is preferably provided which comprises four apparatuses according to the invention, wherein four containers are supplied by a common 400 kW hot water source.

The invention is explained in greater detail with reference to the figures, of which:

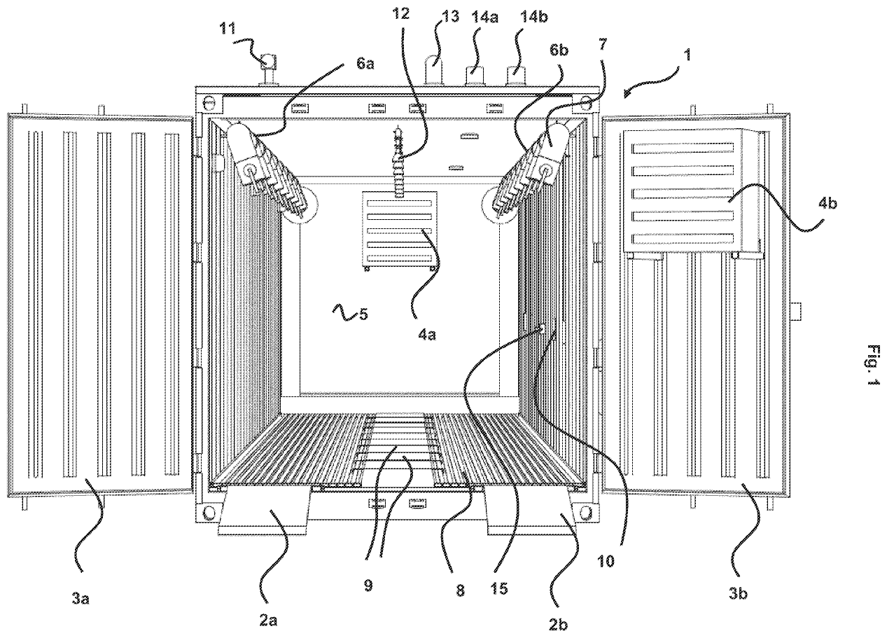

FIG. 1: shows an apparatus according to the invention

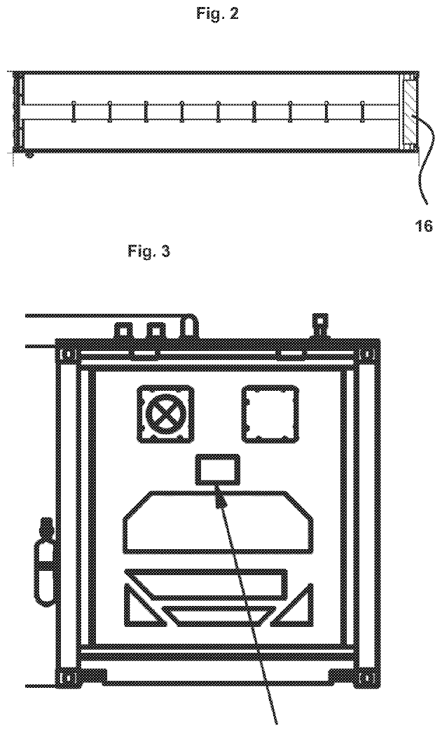

FIG. 2: shows a plan view of an apparatus according to the invention

FIG. 3: shows a rear wall of an apparatus according to the invention from the outside

FIG. 1 shows an apparatus according to the invention in a state with an open loading opening of the insulated sea freight container 1 and extended loading ramps 2a and 2b. A motor vehicle, which is to be heat treated for the purpose of killing pests for example, can roll over the loading ramps 2a, 2b into the container interior. The loading opening can be closed by pivoting the two swing-out doors 3a and 3b after the loading ramps 2a and 2b have been raised. When the loading opening is closed, a respective unit 4a, 4b, in each of which a heat exchanger and a fan are integrated, is arranged on each of the two ends of the interior of the container 1. On the end of the interior of the container 1 opposite the loading opening there is a transverse wall 5, behind which there is a cooling unit (not shown), which has two fans. The cooling unit (not shown in the figure) forms a vertical shaft. Functionally, the rear wall of the cooling unit complements the container wall opposite the loading opening. The transverse wall 5 has through openings (not shown), through which air can flow out of the shaft into the container interior. A respective pipe 6a, 6b is arranged parallel to the longitudinal side in the region of each longitudinal side wall, in the region of the roof of the container interior, which pipe can distribute air in the container interior via a plurality of adjustable outlets 7, which are arranged at regular intervals. A radial fan (not shown) is mounted on the end of each pipe remote from the loading opening. Arranged on the floor of the container are ribs 8, through which there can be a flow of air that is forced into the spaces between the ribs by the fan (not shown) in the cooling unit. The ribs are partially covered by plates 9, with the result that air flowing between the ribs can flow upward only through the openings between the plates. Wired measuring sensors 10 hang from cables on a longitudinal wall of the container interior. Also shown are a hooter 11, a sprinkler system 12, a rotating beacon 13, two operation indicator lamps 14a, 14b and an emergency off switch 15.

The plan view shown in FIG. 2 shows the arrangement of the cooling unit 16 within the refrigerated container.

FIG. 3 shows schematically the wall of the container opposite the loading opening from the outside. This rear wall is complementd functionally by the rear wall of the cooling unit.

1.-15. (canceled)

16. An apparatus for heat treatment of motor vehicles or motor vehicle parts, in particular for the purpose of killing pests, comprising an insulated sea freight container for temperature-controlled transportation, wherein the apparatus comprises at least one fan, optionally a refrigeration unit within the container wherein the refrigeration unit is optionally functional, and at least one heat exchanger.

17. The apparatus as claimed in claim 16, wherein the at least one fan and the at least one heat exchanger are within the container.

18. The apparatus as claimed in claim 16, wherein that the at least one fan and the at least one heat exchanger are integrated in one unit.

19. The apparatus as claimed in claim 16, wherein the container comprises a cooling unit which can be operated by electric current and which comprises a refrigeration unit that is optionally functional, and at least one fan, wherein the cooling unit is arranged on one of two ends of the container, wherein the cooling unit forms a vertical shaft on an inside of the end, wherein a rear wall of the cooling unit functionally complements the wall of the container or is identical therewith, wherein a transverse wall situated opposite the rear wall of the cooling unit has at least one through opening, through which air can flow out of the shaft into the container and/or out of the container into the shaft.

20. The apparatus as claimed in claim 16, configured such that a substance flowing through the heat exchanger is fed in through a first line from outside the container and is discharged through a second line to the outside of the container.

21. The apparatus as claimed in claim 16, wherein the heat exchanger is supplied by a mobile hot water source.

22. The apparatus as claimed in claim 16, wherein the container has a heat exchanger and a fan at each of two ends of the container, wherein the heat output of each of the two heat exchangers is at least 50 kW.

23. The apparatus as claimed in claim 16, wherein at least one pipe parallel to the longitudinal side of the container, having at least two outlets, is configured to distribute air in the container interior.

24. The apparatus as claimed in claim 16, wherein a respective pipe is arranged in the region of the roof in the region of each longitudinal side wall, wherein a radial fan is mounted on the pipe.

25. The apparatus as claimed in 19, wherein partially covered ribs are arranged on the floor of the container and between which air can flow, wherein air flowing between the ribs can flow upward only through particular openings, wherein at least one fan arranged in the cooling unit can force air into spaces between the ribs.

26. The apparatus as claimed in claim 16, further comprising hinged loading ramps at a loading opening, wherein the loading ramps can be detachably secured by at least one clamping device to cooling ribs on the floor of the container.

27. The apparatus as claimed in claim 16, wherein relatively large items of freight or motor vehicles that are to be treated can be heated by combining a plurality of containers lengthwise, transversely and/or vertically.

28. The apparatus as claimed in claim 16, wherein at least one wired measuring sensor is arranged in the container interior.

29. The apparatus as claimed in claim 16, configured to produce an optically or acoustically perceptible signal is produced when all measured values have reached a predetermined limit value and after the expiry of a predetermined minimum time during which all the values remain continuously in a predetermined range.

30. A system comprising four apparatuses, each as claimed in claim 16, wherein four containers are supplied by a common 400 kW hot water source.
